(19) United States
(12) Patent Application Publication
(10) Pub. No.: US 2019/0054060 A1
(43) Pub. Date: Feb. 21, 2019

(54) COMPOSITIONS AND METHODS FOR TREATMENT OF INFLUENZA VIRUS

(71) Applicant: EMERGING VIRAL THERAPEUTICS (HK) LIMITED, Hong Kong (CN)

(72) Inventors: Ivan Fan Ngai HUNG, Hong Kong (CN); Kai Wang Kelvin TO, Hong Kong (CN); Jinxia Anna ZHANG, Hong Kong (CN); Fuk Woo Jasper CHAN, Kowloon (CN); Manson FOK, Hong Kong (CN); Johnson Yiu-Nam LAU, Houston, TX (US); Kwok-Yung YUEN, Pokfulam (CN)

(21) Appl. No.: 16/080,833

(22) PCT Filed: Mar. 1, 2016
(86) PCT No.: PCT/US2016/020292

§ 371 Date: Aug. 29, 2018

(51) Int. Cl. A61K 31/351; A61K 31/19; A61K 31/215; A61P 31/16

(52) U.S. Cl. CPC A61K 31/351; A61K 31/19; A61K 45/06; A61P 31/16; A61K 2300/00; A61K 31/215

(57) ABSTRACT

Therapy for influenza using a combination of a neuraminidase inhibitor, a macrolide antibiotic, and a non-steroidal anti-inflammatory drug has been found to provide improved clinical outcomes and reduced incidence of viral quasispecies compared to conventional treatment with neuraminidase inhibitors alone. Effective treatment schedules are also provided. The drug combination can be used in concert with a proton-pump inhibitor and/or an additional antibacterial antibiotic.

### FIELD OF THE INVENTION

The field of the invention is antiviral compounds and therapies;

### BACKGROUND

Seasonal, pandemic and avian influenza are the most important prevalent viral infections leading to hospitalization and death, particularly among vulnerable populations such as the elderly and individuals with chronic illness. The World Health Organization estimates that seasonal influenza causes 250,000-500,000 deaths worldwide each year. During the 2014-2015 season, the antigenically drifted A/Switzerland/9715293/2013-like H3N2 virus caused major outbreaks in Europe, North America and Asia, resulting in increased morbidity and mortality when compared with the 2013-14 season. Apart from the seasonal influenza, sporadic avian A(H5N1) and A(H7N9) influenza can cause even greater mortality.

Treatment and prophylaxis of influenza is currently limited to neuraminidase inhibitors, such as oseltamivir, zanamivir, peramivir, and laninamivir. Such compounds are most effective when administered early in the infection. Unfortunately patients with severe influenza frequently present clinically after 48 hours from the initial onset of symptoms, rendering neuraminidase treatment less effective.

Other drugs with different modes of action have been used for treating severe influenza infection (Dunning J, Baillie J K, Cao B, Hayden F G, International Severe Acute R, Emerging Infection C. Antiviral combinations for severe influenza. Lancet Infect Dis 2014; 14:1259-70). All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Both in vitro and animal studies have indicated that two FDA-approved drugs, clarithromycin and naproxen, potentially possess antiviral activity (Sawabuchi T, Suzuki S, Iwase K, et al. Boost of mucosal secretory immunoglobulin A response by clarithromycin in pediatric influenza. Respirology 2009; 14:1173-9; Lejal N, Tarus B, Bouguyon E, et al. Structure-based discovery of the novel antiviral properties of naproxen against the nucleoprotein of influenza A virus. Antimicrob Agents Chemother 2013; 57:2231-42). Clarithromycin treatment, for example, can hasten the resolution of symptoms in pediatric influenza patients. Naproxen has been shown to reduce weight loss and pulmonary hemorrhage in mice infected with influenza A virus. Such therapies, however, still fail to produce adequate resolution of influenza symptoms and associated mortality.

Thus, there is still a need for improved compositions and methods for treatment of influenza.

### SUMMARY OF THE INVENTION

The inventive subject matter provides methods and compositions that are effective in treatment of influenza infection. Compositions are provided that include a neuraminidase inhibitor, a macrolide antibiotic, and a non-steroidal anti-inflammatory compound. Treatment protocols are disclosed in which these are provided in combination during an initial treatment period, followed by treatment using a neuraminidase inhibitor outside of such a combination. Additional compounds, such as an antibacterial antibiotic other than the macrolide antibiotic and/or a proton-pump inhibitor can also be provided.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIGS. 1A and 1B show changes in viral count (FIG. 1A) and PSI (FIG. 1B) during initial treatment of influenza-infected individuals with a drug combination of the inventive concept.

FIGS. 2A, 2B, and 2C show changes in NIRV quasispecies composition during treatment of influenza-infected individuals with a drug combination of the inventive concept. FIG. 2A shows the distribution of E119V quasispecies over time. FIG. 2B shows the distribution of N294S quasispecies over time. FIG. 2C shows the distribution of R292K quasispecies over time.

### DETAILED DESCRIPTION

Inventors have found, surprisingly, that use of at least two additional compounds that are not known to have anti-viral activity in combination with a neuraminidase inhibitor shows greater effectiveness in treatment of infections with influenza virus than treatment with the neuraminidase inhibitor alone. One of these additional compounds can be a macrolide antibiotic. Another of these additional compounds can be a non-steroidal anti-inflammatory drug. A treatment schedule that includes an initial dosing period during which a neuraminidase inhibitor, a macrolide antibiotic, and a non-steroidal anti-inflammatory drug is administered followed by a second dosing period during which the neuraminidase inhibitor is administered without the macrolide antibiotic and/or the non-steroidal anti-inflammatory agent has been found to provide a more effective treatment of infection with the influenza virus than the prior art practice of treatment with only neuraminidase inhibitor. Additional pharmaceutical compounds, such as a proton-pump inhibitor and/or an antibiotic other than the macrolide antibiotic can be coadministered during either or both of the initial dosing period and the second dosing period.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

One should appreciate that the compositions and methods disclosed herein provide improved treatment of influenza, resulting in improved clinical outcomes, reduced appearance of resistant viral quasispecies, and reduction in patient mortality relative to the prior art. It should also be appreciated that compositions and methods of the inventive concept can utilize pharmaceutical compounds that have already met regulatory requirements.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Prior art practice for treatment of influenza is administration of neuraminidase inhibitors. While useful in reducing the duration of the illness and the incidence of unfortunate complications resulting from the infection, influenza still results in considerable morbidity and significant mortality. This is particularly true in frail and immunocompromised populations. The inventors have, surprisingly, found that the utility of neuraminidase inhibitors is greatly enhanced by coadministration with a macrolide antibiotic and a non-steroidal anti-inflammatory drug. In preferred embodiments of the inventive concept the neuraminidase inhibitor, macrolide antibiotic, and non-steroidal anti-inflammatory drug are compounds that have already been granted regulatory approval. These compounds can be supplied in combination as a single pharmaceutical composition. Alternatively, all three compounds can be provided as separate compositions. In another embodiment, two of the neuraminidase inhibitor, macrolide antibiotic, and non-steroidal anti-inflammatory drug can be supplied in combination, with the remaining compound supplied as a separate composition. Suitable formulations include pills, capsules, powders (for example, powders suitable for inhalation), and solutions suitable for injection and/or intravenous administration.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

A variety of neuraminidase inhibitors are considered suitable for use in embodiments of the inventive concept. Suitable neuraminidase inhibitors include ethyl (3R,4R,5S)-5-amino-4-acetamido-3-(pentan-3-yloxy)-cyclohex-1-ene-1-carboxylate (oseltamivir), 4S,5R,6R)-5-acetamido-4-carbamimidamido-6-[(1R,2R)-3-hydroxy-2-methoxypropyl]-5,6-dihydro-4H-pyran-2-carboxylic acid (laninamivir), (2R,3R,4S)-4-guanidino-3-(prop-1-en-2-ylamino)-2-((1R,2R)-1,2,3-trihydroxypropyl)-3,4-dihydro-2H-pyran-6-carboxylic acid (zanamivir), and (1S,2S,3S,4R)-3-[(1S)-1-acetamido-2-ethyl-butyl]-4-(diaminomethylideneamino)-2-hydroxy-cyclopentane-1-carboxylic acid (peramivir). Embodiments of the inventive concept can utilize a single neuraminidase inhibitory compound, or can utilize a combination of two or more neuraminidase inhibitory compounds. In some embodiments, the neuraminidase inhibitor that is administered in combination with the macrolide antibiotic and the non-steroidal anti-inflammatory compound can be different from the neuraminidase inhibitor that is administered without the macrolide antibiotic and the non-steroidal anti-inflammatory compound in a two phase dosing schedule. In a preferred embodiment the neuraminidase inhibitor is tamiflu. Neuraminidase inhibitors can be supplied in amounts sufficient to provide from 1 mg to 400 mg per day, and can be administered once a day, twice a day, three times a day, four times a day, more than four times a day, semi-continuously, or continuously. For example, a suitable dosing schedule for a neuraminidase inhibitor can be 50 mg to 100 mg administered orally twice a day.

A variety of macrolide antibiotics are considered suitable for use in embodiments of the inventive concept. Suitable macrolide antibiotics include azithromycin, clarithromycin, erythromycin, fidaxomycin, telithromycin, carbomycin A, josamycin, kitamycin, midecamycin, midecamycin acetate, oleandomycin, solithromycin, spiramycin, troleandomycin, and roxithromycin. In some embodiments, the macrolide antibiotic that is administered in combination with the neuraminidase inhibitor and the non-steroidal anti-inflammatory drug is a combination of two or more macrolide antibiotics. In a preferred embodiment, the macrolide antibiotic is clarithromycin. A macrolide antibiotic can be supplied in amounts sufficient to provide from 100 mg to 4000 mg or more per day, and can be administered once a day, twice a day, three times a day, four times a day, more than four times a day, semi-continuously, or continuously. For example, a suitable dosing schedule for a macrolide antibiotic can be 250 mg to 1000 mg administered orally twice a day.

A variety of non-steroidal anti-inflammatory drugs (NSAIDs) are considered suitable for use in embodiments of the inventive concept. Suitable non-steroidal anti-inflammatory drugs include acetylsalicylic acid, diflunisal, ibuprofen, dexibuprofen, naproxen, indomethacin, tolmetin, sulindac, piroxicam, meloxicam, tenoxicam, mefenamic acid, meclofenamic acid, celecoxib, rofecoxib, valdecoxib, nimesulide, clonixin, licofelone, and flufentamic acid. In some embodiments the non-steroidal anti-inflammatory drug that is administered in combination with the neuraminidase inhibitor and the macrolide antibiotic is a combination of two or more non-steroidal anti-inflammatory drugs. In a preferred embodiment the non-steroidal anti-inflammatory drug is flufentamic acid or naproxen. Dosing of non-steroidal anti-inflammatory drugs is highly dependent upon the particular compound selected. The dosages utilized in embodiments of the inventive concept lie within the therapeutic ranges that are typical for the compound selected when utilized as an analgesic and/or anti-inflammatory. A non-steroidal anti-inflammatory drug can be supplied in amounts sufficient to provide from 5 mg to 2000 mg or more per day, and can be administered once a day, twice a day, three times a day, four times a day, more than four times a day, semi-continuously, or continuously. For example, a suitable dosing schedule for a macrolide antibiotic can be 100 mg to 300 mg administered orally twice a day.

In some embodiments, various accessory pharmaceuticals are provided. For example, a proton-pump inhibitor can be included to offset potential side effects resulting from the administration of a non-steroidal anti-inflammatory drug. Suitable proton-pump inhibitors include esomeprazole, omeprazole, lansoprazole, dexlansoprazole, pantoprazole, rabeprazole, and ilaprazole. In other embodiments, an additional antibacterial antibiotic other than the macrolide antibiotic can be included. Suitable additional antibacterial antibiotics include a penicillin in combination with a penicillinase inhibitor, a cephalosporin, a polymyxin, a rifamycin, a lipiarmycin, a quinolone, a sulfonamide, a lincosamide, a tetracycline, an aminoglycoside, a cyclic lipopeptide, a glycylcline, an oxazolidinone, and a lipiarmycin. In a preferred embodiment the additional antibacterial antibiotic is a penicillin in combination with a penicillinase inhibitor (e.g. amoxicillin in combination with clavulanic acid).

In some embodiments, compositions as described above are provided on a dosing schedule that is suitable to provide improved treatment of influenza over treatment with neuraminidase inhibitors alone. Such a schedule can include a first or initial treatment period during which the afflicted individual is treated with a combination of a neuraminidase inhibitor, a macrolide antibiotic, and a non-steroidal anti-inflammatory drug. This first or initial treatment period can have a duration ranging from 1 day to 14 days. In a preferred embodiment the first or initial treatment period can have a duration ranging from 2 to 4 days. A second treatment period, following the first or initial treatment period, can be provided during which the neuraminidase inhibitor is administered without the macrolide antibiotic and/or the non-steroidal anti-inflammatory drug. Such a second treatment period can have a duration ranging from 1 day to 30 days. In a preferred embodiment the second treatment period can have a duration of from 2 days to 5 days. In some embodiments the first or initial treatment period can constitute the entire course of treatment.

As noted above, an antibacterial antibiotic other than the macrolide antibiotic and/or a proton pump inhibitor can also be administered during the course of treatment. In some embodiments such additional compounds can be administered during the entire course of treatment. In other embodiments such additional compounds can be administered during the first or initial treatment period and not the second treatment period. In still other embodiments such additional compounds can be administered only during the second treatment period.

Effectiveness of the compositions and methods of the inventive concept have been demonstrated in an open-label randomized controlled trial using a combination of clarithromycin, naproxen and oseltamivir as antiviral treatment of influenza A(H3N2) pneumonia in hospitalized patients. The triple combination treatment was associated with significantly lower mortality rates at 30 and 90 days when compared to the oseltamivir control-group. Moreover, the length of hospitalization was also significantly shorter with less frequent requirement of intensive care. These findings corroborated with the significantly faster reduction in viral load, and also a more rapid reduction in PSI between day 0 and 3. The PSI is an objective parameter used to monitor the clinical progress and response to treatment. There was no difference in the baseline characteristics such as comorbidities, presenting symptoms, and duration from symptom onset to treatment between the two groups.

Inventors limited the combination treatment period to the first two days to minimize the potential side effects associated with the NSAID and macrolide. These risks were minimized by administering esomeprazole concurrently. Inventors have found that the viral load is highest on presentation, and therefore early reduction of viral load by using three drugs in the first two days can be beneficial. Adverse events after the short course of combination treatment were uncommon and self-limiting. Only two patients developed a rise in creatinine level, which returned to baseline level 2 days upon completing the treatment.

The appearance of serial changes in non-retroviral integrated RNA viruses (NIRV) quasispecies by pyrosequencing before and after antiviral treatment. These NIRV mutants including E119V, R292K and N294S are responsible for increasing the 50% inhibitory concentrations (IC50) of oseltamivir by >100 fold. The finding of high percentages of low-level NIRV mutant quasispecies at baseline is not surprising because A(H3N2) virus are generally more genetically unstable and intrinsically associated with high frequency of mutation. Nevertheless, the triple antiviral combination of the inventive concept has successfully suppressed the percentage of NIRV quasispecies 24 hours after treatment which was also associated with rapid reduction of viral load and clinical recovery.

It should be appreciated that neuraminidase inhibitor treatment can fail if the patient presents late, has underlying immunocompromising conditions or has developed complication such as pneumonia. Thus a single agent is unlikely to be effective in severe cases, such as during outbreaks of antigenically drifted or shifted influenza, and avian influenza infection, which could be associated with the emergence of antiviral resistant quasispecies. Combination antiviral therapy with compounds having different modes of action can suppress the development of resistant mutants in late presenters.

Despite a relatively short treatment period, combination treatment of the inventive concept was able to suppress the viral replication effectively within 24 hours of treatment initiation. Without wishing to be bound by theory, the inventors believe that each of these three drugs acts on different stages of the viral life cycle. For example, a macrolide antibiotic such as clarithromycin may reduce virus attachment to host cell surface by downregulating the host cell expression of α2,6-linked sialic acid receptor on the cell surface, via the inhibition of NF-κB. Clarithromycin can also inhibit the acidification of the endosomes, which interferes with the uncoating of the virus and fusion of the viral and host cell membrane. NSAIDs, such as naproxen, can inhibit the replication process by interfering with the binding of the viral RNA and nucleoprotein. A neuraminidase inhibitor, such as oseltamivir, can act at the last step by inhibiting release of progeny virus from the host cell surface. Outside the host cell, the antiviral combination can also inhibit the virus from getting into the cells. Oseltamivir is known to prevent the virus from reaching the host cell by affecting its trafficking through the sialic acid rich mucus layer in the airway. Clarithromycin can enhance the secretion of specific mucosal IgA against influenza virus by increasing the airway IgA class switching. This can counteract the attenuation effect of oseltamivir on production of specific secretory IgA against influenza virus in mice. Furthermore the anti-inflammatory effect of naproxen and clarithromycin can also contribute to the better outcome of the combination-treatment group.

### Examples

Study Design: This was a phase 2b/3, single-center clinical trial with randomized treatment-group assignments and open-label treatment. Combination oral treatment with clarithromycin and naproxen plus the usual antiviral treatment of oseltamivir, was compared to that of oseltamivir alone as control in patients hospitalized for laboratory-confirmed influenza A(H3N2) influenza infection with pneumonia. The study was designed on a difference of 18% in mortality in patients with severe influenza, when treated with combination vs. oseltamivir alone therapy12; sample size was calculated to be 93 patients per group (one-sided alpha level of 0.05, with 80% power).

The study protocol was approved by the institutional review board of the University of Hong Kong and Hospital Authority, and is registered with the ISRCTN, number 11273879. All patients provided written informed consent before randomization.

Members of the study committee and the local investigators designed the study, collected and analyzed the data, wrote the manuscript, and made the decision to submit the manuscript for publication. The study sponsors were not involved in the study design, study conduct, protocol review, or manuscript preparation or review.

Patients: The study was conducted in Queen Mary Hospital, a teaching hospital for the University of Hong Kong Medical School between February and April 2015. The main inclusion criteria were patients ≥18 years of age, auditory temperature ≥38° C. with at least one of the following symptoms (cough, sputum production, sore-throat, nasal discharge, myalgia, headache or fatigue) upon admission, symptom duration <72 hours, laboratory-confirmed A(H3N2) influenza, radiological changes of pulmonary infiltrate by chest radiography or computerized tomography, and clinically required hospitalization. Diagnosis of A(H3N2) infection was confirmed by reverse transcription-polymerase chain reaction (RT-PCR) in the nasopharyngeal aspirate (NPA). Initiation of antiviral treatment had to be commenced within 24 hours after hospital admission. Patients with history of allergy to oseltamivir, clarithromycin, non-steroidal anti-inflammatory drugs (NSAIDs) and beta-lactam antibiotics, or moderate renal impairment (creatinine clearance <30 mL/min) were excluded. Detailed inclusion and exclusion criteria are listed in the study protocol.

Randomization and Intervention: Recruited patients were assigned into one of two groups by simple randomization with no stratification: the study group, a triple combination of clarithromycin 500 mg, naproxen 200 mg and oseltamivir 75 mg twice daily for 2 days, followed by 3 days of oseltamivir 75 mg twice daily; or the control-group, oseltamivir 75 mg twice daily for 5 days (ratio 1:1). All patients received 5 days of oral amoxicillin-clavulanate 1 g twice daily for empirical treatment of pneumonia, and esomeprazole 20 mg daily for prevention of NSAID-induced gastropathy. Each enrolled patient was assigned a serial number, and randomization lists were computer-generated in blocks of two by the study nurse. Randomization lists were then used to assign each serial number to one of the study groups. All subjects were followed-up for 90 days after antiviral treatment.

Outcome and Safety Measures: The primary outcome was mortality at 30 days and the secondary outcomes were mortality at 90 days, serial changes in the NPA viral titer, percentage change of neuraminidase inhibitor-resistant A(H3N2) virus (NIRV) quasispecies, the pneumonia severity index (PSI) from day 1 to 4 after antiviral treatment, and length of hospitalization. The PSI was determined daily for all recruited patients from admission till discharge, transfer to a convalescent hospital or death. Arterial blood gas was measured in patients who required respiratory support.

Clinical, Virological and Radiological Assessment: The diagnosis of A(H3N2) influenza was confirmed when the M and H3 genes were tested positive by RT-PCR in NPA specimens taken on admission. Clinical findings including history and physical examination, oximetric measurement, hematological, biochemical, radiological, and microbiological investigation results were prospectively entered into a predesigned database. Viral load was determined using quantitative RT-PCR. NPA were collected every day if possible from admission till discharge, transfer to a convalescent hospital or death. Percentage of NIRV quasispecies including E119V, R292K and N294S mutants were determined by pyrosequencing analysis in any NPA specimens containing a viral load of >1000 copies/mL, a level sufficient for accurate pyrosequencing analysis (Supplementary Appendix). Admission to intensive care unit, requirement of oxygen, mechanical ventilatory, bilevel positive airway pressure (BiPAP) and continuous positive airway pressure (CPAP) support were documented.

The NPA upon admission was assessed by NxTAG™ Respiratory Pathogen Panel for co-infection with Mycoplasma pneumoniae, Chlamydophilia pneumonia, and Legionella pneumophila. Samples tested included blood, sputum or endotracheal aspirates, and urine bacteriologically, as clinically indicated.

Statistical analysis: Clinical and virological characteristics were compared. Fisher's exact test and X2 test were used for categorical variables where appropriate, whereas Mann-Whitney U-test was used for continuous variables, including comparison for the percentage of NIRV quasispecies. The 30-day and 90-day mortality was compared by Cox-regression. The reduction of viral load, PSI and length of hospital stay was compared by linear regression. A p-value <0.05 represents significant difference.

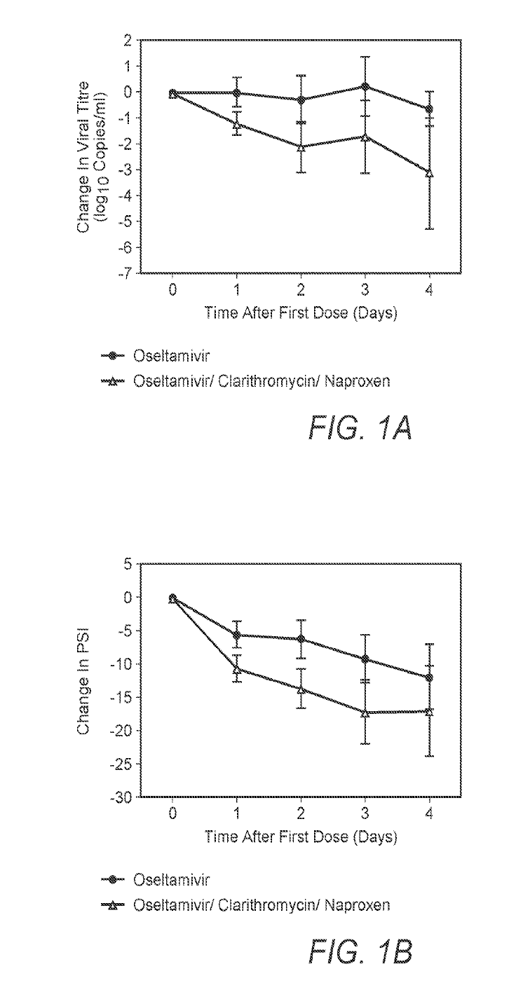

Results: Between February and April 2015, 278 hospitalized patients with A(H3N2) infection were screened (FIG. 1). Of the 278 patients screened, 61 patients refused to give informed consent and 217 (78.1%) were enrolled after virological confirmation by RT-PCR and radiological confirmation of pulmonary infiltrate. Of these, 107 (49.3%) were randomly assigned to receive the triple combination treatment. All recruited patients completed the study and the outcome was analyzed by intention-to-treat. There was no difference in patients' baseline demographics, comorbidities, presenting clinical features, laboratory and chest radiographic findings between the two groups (Tables 1A to 1E and 2). None of the enrolled patients received neuraminidase inhibitor treatment before collection of the first NPA sample or in past 12 months. There was no difference in the number of respiratory failures, NPA viral load, percentage of NIRV quasispecies and PSI at baseline between the two groups (Table 2). Two patients from the combination-treatment-group developed a rise in serum creatinine to 120 μmol/L and 132 μmol/L respectively from baseline 3 days after treatment. Creatinine level returned to their normal baseline levels of 102 μmol/L and 106 μmol/L respectively within 2 days after completion of naproxen and clarithromycin treatment. No patients developed gastrointestinal side effects during the study period.

Ten patients succumbed during the 30-day follow-up period of which 1 (0.9%) were from the combination-treatment-group, comparing to 9 patients (8.2%) who succumbed in the oseltamvir control-group (Table 2). The primary outcome in Kaplan-Meier analysis by log-rank test showed that participants who received the combination treatment had significantly lower 30-day mortality (p=0.036; hazard ratio [HR]:0.11; 95% confidence interval [C.I.]:0.01-0.86) and 90-day mortality (p=0.025; HR: 0.18; 95% C.I.: 0.04-0.81) than the control (Supplementary FIG. 51). The median time of death was 14 days [interquartile range (IQR): 2 to 26 days] after symptom onset. Length of hospitalization was also shorter in the combination-treatment-group (p<0.0001; HR: 0.39; 95% C.I.: 0.49-0.65).

There was no difference in the baseline viral load and PSI between the two groups (Table 1A to Table 1E). Reduction of viral load was significantly faster in the combination-treatment-group than the control-group from day 1 to 4, especially for the first 2 days of treatment (FIG. 1A). Statistical treatment of the data presented in FIG. 1A can be found in Table 3. Reduction in viral load occurred most rapidly in the combination-treatment-group between day 0 and day 1 [−1.18 log 10 copies/mL; 95% confidence interval (95% C.I.−1.63 to −0.74 log₁₀ copies/mL; p=0.001)] and day 2 [−2.1 log₁₀ copies/mL; (95% C.I.−3.08 to −1.12 log₁₀ copies/mL; p=0.007)]. In correlation with the changes in viral load, reduction in PSI was also significantly faster in the combination-treatment-group than the control-group for the first 3 days of treatment (FIG. 1B). Statistical treatment of the data presented in FIG. 1B can be found in Table 4. Reduction in PSI occurred most rapidly in the combination-treatment-group between day 0 and 1 [−10.61; (95% C.I.−12.66 to −8.55; p=0.001)] and between day 1 and 2 [(−13.65; (95% C.I.−16.56 to −10.75; p<0.0001)].

TABLE 1A

| | Oseltamivir/ | | | | |
|---|---|---|---|---|---|
| | Clarithromycin/ | | | | |
| | Naproxen | | Oseltamivir | | |
| | (n = 107) | | (n = 110) | | p value |
| Demographics | | | | | |
| Median age in years (IQR) | 80 | (72-85) | 81.5 | (71-87.3) | 0.27 |
| Sex (male:female) | 63:44 | | 53:57 | | 0.11 |
| Smoker (current or ex- | 30 | (28) | 24 | (21.8) | 0.29 |
| smoker) - no.(%) | | | | | |
| Elderly home resident - | 24 | (22.4) | 34 | (30.9) | 0.18 |
| no. (%) | | | | | |
| Previous neuraminidase | 0 | (0) | 0 | (0) | na |
| inhibitors - no. (%) | | | | | |
| Past Medical | | | | | |
| History - no. (%) | | | | | |
| Good past health | 49 | (45.8) | 49 | (44.5) | 0.85 |
| Cardiovascular disease | 25 | (23.4) | 25 | (22.7) | 0.91 |
| Pulmonary disease | 19 | (17.8) | 14 | (12.7) | 0.30 |
| Cerebrovascular disease | 18 | (16.8) | 23 | (20.9) | 0.44 |
| Hepatitis B carrier | 3 | (2.8) | 1 | (0.9) | 0.30 |
| Renal disease | 3 | (2.8) | 4 | (3.6) | 0.73 |
| Malignancy | 12 | (11.2) | 15 | (13.6) | 0.59 |

TABLE 1B

| | Oseltamivir/ | | |
|---|---|---|---|
| | Clarithromycin/ | | |
| | Naproxen | Oseltamivir | |
| Presenting symptoms - no. (%) | (n = 107) | (n = 110) | p value |
| Fever | 111 (100) | 110 (100) | na |
| Cough | 84 (78.5) | 81 (73.6) | 0.40 |
| Sputum | 69 (64.5) | 65 (59.1) | 0.41 |
| Rhinorrhea | 32 (29.9) | 25 (22.7) | 0.23 |
| Sore throat | 9 (8.4) | 9 (8.2) | 0.95 |
| Chills | 10 (9.3) | 9 (8.2) | 0.76 |
| Wheezing | 10 (9.3) | 11 (10) | 0.87 |
| Headache | 5 (4.7) | 3 (2.7) | 0.44 |
| Dizziness | 12 (11.2) | 9 (8.2) | 0.45 |
| Dyspnea | 48 (44.9) | 57 (51.8) | 0.31 |
| Pleuritic chest pain | 13 (12.1) | 11 (10) | 0.63 |
| Vomiting | 11 (10.3) | 13 (11.8) | 0.74 |
| Diarrhea | 5 (4.7) | 5 (4.5) | 0.96 |

TABLE 1C

| | Oseltamivir/ | | | | |
|---|---|---|---|---|---|
| | Clarithromycin/ | | | | |
| Initial physical | Naproxen | | Oseltamivir | | p |
| examination findings | (n = 107) | | (n = 110) | | value |
| Altered mental | 18 | (16.8) | 19 | (17.3) | 0.93 |
| status - no. (%) | | | | | |
| Median systolic blood | 151 | (135-169) | 149.5 | (133-167) | 0.65 |
| pressure (IQR, mmHg) | | | | | |
| Median pulse rate | 94 | (81-108) | 93 | (80-109) | 0.97 |
| (IQR, /min) | | | | | |
| Median respiratory | 18 | (16-20) | 18 | (16-20) | 0.78 |
| rate (IQR, /min) | | | | | |
| Median temperature | 38.3 | (37.6-38.9) | 38 | (37.2-38.7) | 0.06 |
| (IQR, >38° C.) | | | | | |

TABLE 1D

| | Oseltamivir/ | | | | |
|---|---|---|---|---|---|
| Initial laboratory | Clarithromycin/ | | | | |
| findings - median | Naproxen | | Oseltamivir | | p |
| (IQR) | (n = 107) | | (n = 110) | | value |
| Total white blood | 7.9 | (5.8-10.2) | 7 | (6.0-10.7) | 0.64 |
| cell (×10⁹/L) | | | | | |
| Neutrophil (×10⁹/L) | 6.0 | (4.1-8.3) | 5.8 | (4.5-8.6) | 0.95 |
| Lymphocyte (×10⁹/L), | 0.9 | (0.6-1.3) | 0.9 | (0.5-1.3) | 0.76 |
| Hemoglobin (g/dL) | 11.9 | (10-13.5) | 12 | (10.5-13.2) | 0.63 |
| Hematocrit | 0.35 | (0.3-0.4) | 0.36 | (0.32-0.4) | 0.43 |
| Alanine transaminase | 20 | (15-27) | 19.5 | (14-32) | 0.82 |
| (IU/L) | | | | | |
| Aspartate transaminase | 29 | (23-40) | 31.5 | (23.8-44) | 0.31 |
| (IU/L) | | | | | |
| Alkaline phosphatase | 71 | (54-89) | 75 | (57.5-93) | 0.38 |
| (IU/L) | | | | | |
| Sodium (mmol/L) | 137 | (133-140) | 137 | (135-140) | 0.07 |
| Creatinine (μmol/L) | 83 | (69-99) | 86 | (66-112.3) | 0.35 |
| Urea (mmol/L) | 5.6 | (4.4-7.2) | 5.6 | (4.4-9.8) | 0.40 |
| Glucose (mmol/L) | 6.3 | (5.0-7.6) | 6 | (5.0-7.6) | 0.86 |
| Arterial pH | 7.44 | (7.40-7.48) | 7.40 | (7.40-7.45) | 0.32 |
| (67 patients) | | | | | |
| Arterial PO₂ (kPa) | 10.6 | (8.8-13.2) | 12.2 | (9.8-17.8) | 0.16 |
| (67 patients) | | | | | |

TABLE 1E

| | Oseltamivir/ | | | | |
|---|---|---|---|---|---|
| | Clarithromycin/ | | | | |
| | Naproxen | | Oseltamivir | | |
| | (n = 107) | | (n = 110) | | p value |
| Initial radiological | | | | | |
| findings - no. (%) | | | | | |
| Infiltrate | 96 | (89.7) | 97 | (88.2) | 0.80 |
| Consolidation | 9 | (8.4) | 8 | (7.3) | 0.80 |
| Pleural effusion | 2 | (1.9) | 5 | (4.5) | 0.27 |
| Baseline viral load | 7.2 | (5.4-8.2) | 7.6 | (6.1-8.3) | 0.42 |
| (mean log₁₀ copies/ml; | | | | | |
| 95% C.I.) | | | | | |
| Baseline pneumonia | 95 | (81-112) | 96.5 | (85.8-113) | 0.74 |
| severity index | | | | | |
| (mean; 95% C.I.) | | | | | |

TABLE 3

| | Days after first dose | | | | |
|---|---|---|---|---|---|
| | 0 | 1 | 2 | 3 | 4 |
| Oseltamivir | 110 | 51 | 21 | 12 | 6 |
| group (n) | | | | | |
| Mean change | | 0.02 | −0.26 | 0.25 | −0.62 (−1.28 |
| (95% CI) viral | | (−0.55 | (−1.18 | (−0.90 | to 0.04) |
| titer log₁₀ | | to 0.59) | to 0.66) | to 1.39) | |
| copies/mL | | | | | |
| from baseline | | | | | |
| Oseltamivir/ | 107 | 55 | 23 | 9 | 8 |
| Clarithromycin/ | | | | | |
| Naproxen | | | | | |
| group (n) | | | | | |
| Mean change | | −1.18 | −2.10 | −1.68 | −3.11 (−5.24 |
| (95% CI) viral | | (−1.63 | (−3.08 | (−3.09 | to −0.99) |
| titer log₁₀ | | to −0.74) | to −1.12) | to −0.28) | |
| copies/mL | | | | | |
| from baseline | | | | | |
| p-value | | 0.001 | 0.007 | 0.003 | 0.038 |

TABLE 4

| | Days after first dose | | | | |
|---|---|---|---|---|---|
| | 0 | 1 | 2 | 3 | 4 |
| Oseltamivir | 110 | 110 | 95 | 61 | 31 |
| group (n) | | | | | |
| Mean change | | −5.54 (−7.51 to | −6.26 (−9.14 | −9.18 (−12.84 | −11.94 |
| (95% CI) in | | −3.57) | to −3.39) | to −5.52) | (−16.8 to −7.07) |
| PSI from | | | | | |
| baseline | | | | | |
| Oseltamivir/ | 107 | 107 | 89 | 43 | 20 |
| Clarithromycin/ | | | | | |
| Naproxen | | | | | |
| group (n) | | | | | |
| Mean change | | −10.61 (−12.66 | −13.65 (−16.56 | −17.09 (−21.86 | −17.00 (−23.81 |
| (95% CI) in | | to −8.55) | to −10.75) | to −12.33) | to −10.19) |
| PSI from | | | | | |
| baseline | | | | | |
| p-value | | 0.001 | <0.0001 | 0.008 | 0.21 |

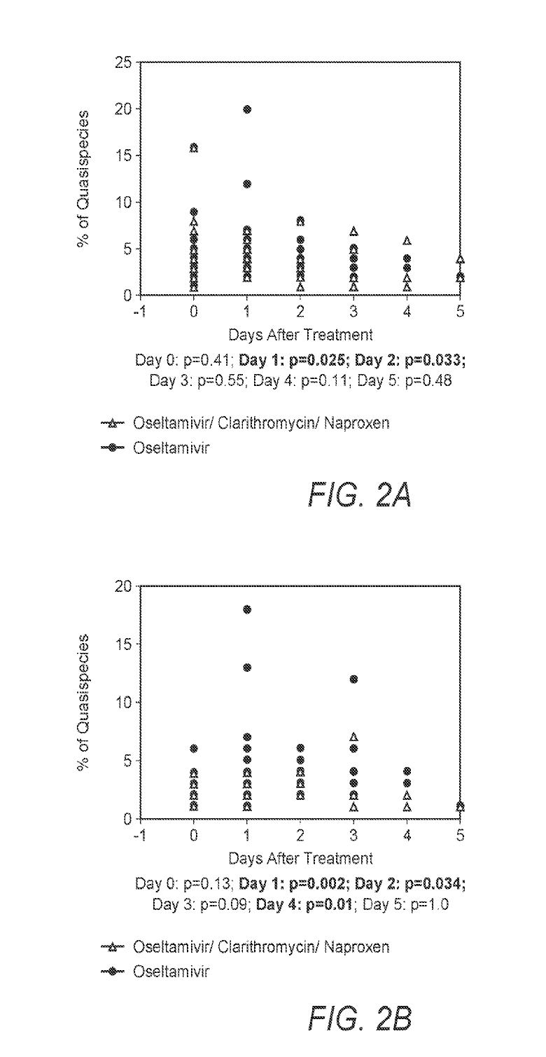

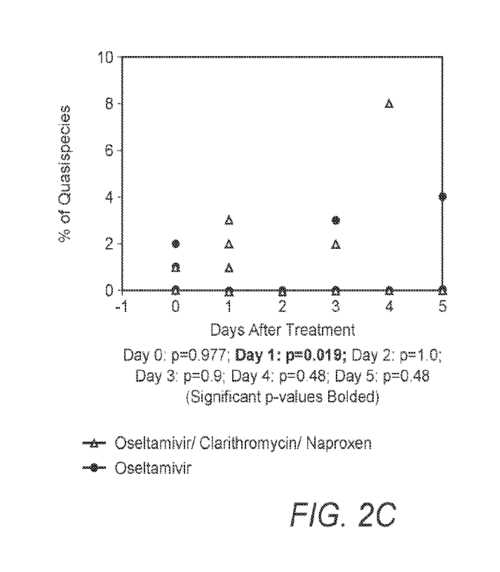

Pyrosequencing analysis of 72 patients with available serial NPA samples containing sufficient viral load (36 patients from each group) demonstrated a detectable percentage of NIRV quasispecies at baseline (median; IQR), E119V: 3% (2-4%) and N294S: 3% (2-3%). NIRV identified as R292K quasispecies were relatively rare. Only 4 samples had R292K quasipecies at baseline, including one sample with 2% and 3 samples with 1%. There was no difference in the percentage of these 3 types of NIRV quasispecies between the two groups at baseline (FIGS. 2A and 2B). Percentages of E119V and N294S resistant quasispecies were significantly lower on post-treatment day 1 and day 2 in the combination-treatment-group when compared to the control-group (p<0.05) (FIGS. 2A and 2B). Percentage of R292K resistant quasispecies was significantly lower only on day 1 (p=0.019) in the combination-treatment-group when compared to control-group (FIG. 2C).

Bacterial co-infections upon admission were uncommon with no difference between the two groups (Table 2). None of the patients were diagnosed to have atypical bacterial infections. Three patients required mechanical ventilator support of which two were from the control-group. Fifteen and eleven patients required BiPAP and CPAP ventilator support respectively with no difference between the two groups. Six patients from each group developed nosocomial infections. There was no difference in the readmission rate within 30 days after discharge between the two groups.

TABLE 2

| | Oseltamivir/ | | | | |
|---|---|---|---|---|---|
| | Clarithromycin/ | | | | |
| | Naproxen | | Oseltamivir | | |
| | (n = 107) | | (n = 110) | | p value |
| Days of symptoms before | 2 | (1-3) | 1 | (1-3) | 0.17 |
| starting antiviral treatment | | | | | |
| (median; IQR) | | | | | |
| Respiratory support upon | | | | | |
| admission - no. (%) | | | | | |
| Oxygen | 42 | (39.2) | 36 | (32.7) | 0.32 |
| Mechanical ventilation | 2 | (1.8) | 1 | (0.9) | 0.55 |
| BiPAP | 7 | (6.5) | 8 | (7.3) | 0.83 |
| CPAP | 6 | (5.6) | 4 | (3.6) | 0.49 |
| Complications - no. (%) | | | | | |
| Bacterial co-infection upon | 5 | (4.7) | 3 | (2.7) | 0.45 |
| presentation† | | | | | |
| Ventilator associated pneumonia | 0 | (0) | 1 | (0.9) | 0.32 |
| Nosocomial infection‡ | 6 | (5.6) | 6 | (5.5) | 0.96 |
| Admission to ICU - no. (%) | 1 | (0.9) | 3 | (2.7) | 0.33 |
| Length of hospitalization | 3 | (1-9) | 3 | (2-16) | 0.03 |
| (median; IQR) | | | | | |
| Readmission ≤30 days from | 20 | (18.7) | 19 | (17.3) | 0.79 |
| discharge - no. (%) | | | | | |
| Mortality - no. (%) | | | | | |
| 30-day | 1 | (0.9) | 9 | (8.2) | 0.011 |
| 90-day | 2 | (1.9) | 11 | (10) | 0.012 |

*BiPAP: bilevel positive airway pressure;
CPAP: continuous positive airway pressure;
IQR: interquartile range;
ICU: intensive care unit;
†Streptococcus pneumoniae in blood culture (n = 1 in combination-treatment-group); Methicillin sensitive Staphylococcus aureus in blood culture (n = 1 in the control-group). Haemophilus influenzae in sputum culture of 5 patients (n = 3 in combination-treatment-group and n = 2 in control-group); Significant bacteriuria for Streptococcus agalactiae (n = 1 in combination-treatment-group).
‡Pseudomonas aeruginosa in sputum culture (n = 5 in combination-treatment-group; n = 4 in control-group); Endotracheal aspirate positive for mixed infection by methicillin resistant Staphylococcus aureus, Klebsiella spp, and Pseudomonas aeruginosa in control group (n = 1); Positive urine culture was for Enterococcus fecalis (n = 1from each group).

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1.-40. (canceled)

41. A composition for treating infection with an influenza virus, comprising:
a neuraminidase inhibitor;
a macrolide antibiotic; and
a non-steroidal anti-inflammatory drug.

42. The composition of claim 1, wherein the neuraminidase inhibitor is selected from the group consisting of ethyl (3R,4R,5S)-5-amino-4-acetamido-3-(pentan-3-yloxy)-cyclohex-1-ene-1-carboxylate (oseltamivir), 4S,5R,6R)-5-acetamido-4-carbamimidamido-6-[(1R,2R)-3-hydroxy-2-methoxypropyl]-5,6-dihydro-4H-pyran-2-carboxylic acid (laninamivir), (2R,3R,4S)-4-guanidino-3-(prop-1-en-2-ylamino)-2-((1R,2R)-1,2,3-trihydroxypropyl)-3,4-dihydro-2H-pyran-6-carboxylic acid (zanamivir), and (1S,2S,3S,4R)-3-[(1S)-1-acetamido-2-ethyl-butyl]-4-(diaminomethylideneamino)-2-hydroxy-cyclopentane-1-carboxylic acid (peramivir).

43. The composition of claim 1, wherein the macrolide antibiotic is selected from the group consisting of azithromycin, clarithromycin, erythromycin, fidaxomycin, telithromycin, carbomycin A, josamycin, kitamycin, midecamycin, midecamycin acetate, oleandomycin, solithromycin, spiramycin, troleandomycin, and roxithromycin.

44. The composition of claim 1, wherein the non-steroidal anti-inflammatory drug is selected from the group consisting of acetylsalicylic acid, diflunisal, ibuprofen, dexibuprofen, naproxen, indomethacin, tolmetin, sulindac, piroxicam, meloxicam, tenoxicam, mefenamic acid, meclofenamic acid, celecoxib, rofecoxib, valdecoxib, nimesulide, clonixin, licofelone, and flufentamic acid.

45. The composition of claim 1, further comprising a proton-pump inhibitor.

46. The composition of claim 5, wherein the proton-pump inhibitor is selected from the group consisting of omeprazole, lansoprazole, dexlansoprazole, pantoprazole, rabeprazole, and ilaprazole.

47. The composition of claim 1, further comprising an anti-bacterial antibiotic that is not the macrolide antibiotic.

48. The composition of claim 7, wherein the anti-bacterial antibiotic is selected from the group consisting of a penicillin, a penicillin in combination with a penicillinase inhibitor, a cephalosporin, a polymyxin, a rifamycin, a lipiarmycin, a quinolone, a sulfonamide, a lincosamide, a tetracycline, an aminoglycoside, a cyclic lipopeptide, a glycylcline, an oxazolidinone, and a lipiarmycin.

49. A method of treating an infection with an influenza virus, comprising:
providing a neuraminidase inhibitor, a macrolide antibiotic, and a non-steroidal anti-inflammatory drug; and
administering the neuraminidase inhibitor, the macrolide antibiotic, and the non-steroidal anti-inflammatory drug to an individual on a schedule effective in reducing mortality due to influenza.

50. The method of claim 9, wherein the schedule comprises administering the composition for a first period of time followed by administration of the neuraminidase inhibitor for a second period of time.

51. The method of claim 9, wherein the macrolide antibiotic is selected from the group consisting of azithromycin, clarithromycin, erythromycin, fidaxomycin, telithromycin, carbomycin A, josamycin, kitamycin, midecamycin, midecamycin acetate, oleandomycin, solithromycin, spiramycin, troleandomycin, and roxithromycin.

52. The method of claim 9, wherein the non-steroidal anti-inflammatory drug is selected from the group consisting of acetylsalicylic acid, diflunisal, ibuprofen, dexibuprofen, naproxen, indomethacin, tolmetin, sulindac, piroxicam, meloxicam, tenoxicam, mefenamic acid, meclofenamic acid, celecoxib, rofecoxib, valdecoxib, nimesulide, clonixin, licofelone, and flufentamic acid.

53. The method of claim 9, further comprising a step of administering a proton-pump inhibitor.

54. The method of claim 13, wherein the proton-pump inhibitor is selected from the group consisting of omeprazole, lansoprazole, dexlansoprazole, pantoprazole, rabeprazole, and ilaprazole.

55. The method of claim 9, further comprising a step of administering an anti-bacterial antibiotic that is not the macrolide antibiotic.

56. The method of claim 15, wherein the anti-bacterial antibiotic is selected from the group consisting of a penicillin, a penicillin in combination with a penicillinase inhibitor, a cephalosporin, a polymyxin, a rifamycin, a lipiarmycin, a quinolone, a sulfonamide, a lincosamide, a tetracycline, an aminoglycoside, a cyclic lipopeptide, a glycylcline, an oxazolidinone, and a lipiarmycin.

57. A kit for treating disease resulting from infection with an influenza virus, comprising:
a neuraminidase inhibitor;
a macrolide antibiotic;
a non-steroidal anti-inflammatory drug; and
instructions for administering the neuraminidase inhibitor, the macrolide antibiotic, and the non-steroidal anti-inflammatory drug to an individual on a schedule effective in reducing mortality due to influenza, wherein the schedule comprises administering the neuraminidase inhibitor, the macrolide antibiotic, and the non-steroidal anti-inflammatory drug for a first period of time followed by administration of only the neuraminidase inhibitor for a second period of time.

58. The kit of claim 17, wherein the macrolide antibiotic is selected from the group consisting of azithromycin, clarithromycin, erythromycin, fidaxomycin, telithromycin, carbomycin A, josamycin, kitamycin, midecamycin, midecamycin acetate, oleandomycin, solithromycin, spiramycin, troleandomycin, and roxithromycin, and wherein the non-steroidal anti-inflammatory drug is selected from the group consisting of acetylsalicylic acid, diflunisal, ibuprofen, dexibuprofen, naproxen, indomethacin, tolmetin, sulindac, piroxicam, meloxicam, tenoxicam, mefenamic acid, meclofenamic acid, celecoxib, rofecoxib, valdecoxib, nimesulide, clonixin, licofelone, and flufentamic acid.

59. The kit of claim 17, wherein the composition further comprising a proton-pump inhibitor, wherein the proton-pump inhibitor is selected from the group consisting of omeprazole, lansoprazole, dexlansoprazole, pantoprazole, rabeprazole, and ilaprazole.

60. The kit of claim 17, wherein the composition further comprising an anti-bacterial antibiotic that is not the macrolide antibiotic, wherein the anti-bacterial antibiotic is selected from the group consisting of a penicillin, a penicillin in combination with a penicillinase inhibitor, a cephalosporin, a polymyxin, a rifamycin, a lipiarmycin, a quinolone, a sulfonamide, a lincosamide, a tetracycline, an aminoglycoside, a cyclic lipopeptide, a glycylcline, an oxazolidinone, and a lipiarmycin.
